(19) United States
(12) Patent Application Publication
(10) Pub. No.: US 2020/0330704 A1
(43) Pub. Date: Oct. 22, 2020

(54) NOZZLE BODY AND SPRAYING DEVICE

(71) Applicant: WERRTA GmbH Düsen- und Zerstäubungstechnik, Berlin (DE)

(72) Inventor: Rudiger RENTSCH, Berlin (DE)

(21) Appl. No.: 16/753,932

(22) PCT Filed: Oct. 5, 2018
(86) PCT No.: PCT/EP2018/077158

§ 371 Date: Apr. 6, 2020

(30) Foreign Application Priority Data
Oct. 6, 2017 (DE) 20 2017 005 165.6

(51) Int. Cl. A61M 11/00; C03C 3/06; A61M 15/00; B05B 1/34; C03C 15/00; C03C 23/00; B65D 83/64; B65D 83/28; B65D 83/20

(52) U.S. Cl. CPC A61M 11/003; C03C 3/06; A61M 15/009; A61M 11/007; B05B 1/3426; A61M 15/08; C03C 23/0025; B65D 83/64; B65D 83/28; B65D 83/205; C03C 2201/02; C03C 15/00

(57) ABSTRACT

By means of selective laser exposure and subsequent etching away of the exposed regions (selective laser-induced etching), a cylindrical-conical cavity (15) having a mainly tangential fluid inlet (16) and axial fluid outlet (17) is formed in the quartz glass nozzle body (14) having a generally cylindrical shape.

The present invention relates to a nozzle body, in particular also for the spray head of a spraying device, for example a spray can.

Nozzle bodies are frequently used in the art when fluids such as gases, liquids and aerosols are to be atomized, i.e. converted into an aerosol, from a supply line or a storage container, and discharged in doses, i.e. with a flow rate within a predetermined range. The respective fluid or fluid mixture is supplied to the nozzle body through one or more inlet openings from the corresponding supply line or receiver and leaves it through one or more outlet channels. A wide range of applications are known to the skilled person from spray systems such as inhalers or simple spray cans, injection systems etc.

Sufficiently small droplets, as may be required in particular for achieving respirable properties in the medical field, can only be produced if the structures in the spray head are also sufficiently small. The reduction of the fluid throughput at a given pressure gradient also requires correspondingly small structures in the spray head.

In addition to the droplet size achievable and the resulting flow rate, the breadth of the droplet size distribution is also a parameter which is, to a great extent, determined by the geometric design of the spray head.

The design of the spray head geometry has been limited so far for reasons of available manufacturing technology, so that not every geometry that is theoretically suitable for setting the above parameters can be produced with any desired degree of precision at economic conditions. In particular, for conventional spray heads, the surface quality inside the outlet channels forming the nozzles is usually insufficient, so that the pressure loss in the outlet channels is often too high and the desired atomization performance is not achieved.

While in stationary inhalation systems the controllability of the pressure and the, in principle, availability of even very high fluid pressures over a longer period of time offer additional possibilities for adjusting the above parameters, in the case of spray cans the limited tank pressure, which decreases with increasing time of use, must be sufficient.

Therefore, possible applications of conventional spray cans are limited. For example, medical sprays are generally only suitable for administering individual short spray bursts, a longer duration of continuous spraying is limited by the supply of spray material and propellant, as the dosage of conventional sprays is rather coarse for the above reasons, i.e. reducing the discharged flow of spray material for yielding a longer spraying duration is generally not possible for the user.

Generally, the design of conventional spray heads, especially for sprays with relatively low pressures, thus often fails because a desired droplet size distribution and/or a desired volume flow cannot be set precisely enough.

WO 94/07607 A1 describes manufacturing a spray head for a high-pressure inhaler by processing individual layers with laser and then laminating them together. This process is extremely complex. WO 2009/090084 A1 discloses the manufacture of another spray head for a high-pressure inhaler by deep-drawing a metal sheet into which openings have previously been introduced by laser drilling. Using this manufacturing process it is difficult to achieve sufficient manufacturing precision. WO 2016/075433 A1 discloses production of a spray head as an injection-moulded plastic part, wherein the nozzles are produced by means of laser drilling. Again, the possible manufacturing precision is hardly sufficient to set a desired droplet size distribution by exact selection of the nozzle geometry.

In view of the limitations applying to the use of conventional nozzle bodies, especially in spray cans, inhalers and atomizer pumps, the present invention is based on the problem of at least reducing the disadvantages of the nozzle bodies known from the prior art. In particular, it is an underlying object of the present invention to extend the range of available geometries, in the finest range, for nozzle bodies that can be produced at reasonable economic expense.

According to one aspect of the present invention, this object is solved by a nozzle body which comprises a conical or cylindrical-conical cavity through which fluid can flow and which has at least one fluid inlet which is predominantly tangential relative to the cavity jacket and one fluid outlet which is substantially axial relative to the cavity jacket, the nozzle body being formed in one piece from a glass material, and the smallest diameter of the fluid inlet and the smallest diameter of the fluid outlet each being 100 micrometers or less.

Therein, the smallest diameter is the distance from inner surface to inner surface of the fluid inlet or fluid outlet along a straight line orthogonally intersecting the connecting line of the centroids of the two flowed-through areas spanned by the edges of the fluid inlet or outlet, respectively.

In this context, predominantly tangential is understood to mean in particular that the connecting line of the centers of gravity of the two flowed-through areas, which are spanned by the edges of the fluid inlet, has a greater tangential component than radial component at its point of intersection with the jacket surface of the cavity. At this intersection, the angle of the line connecting the two areas relative to a tangent line orthogonal to the longitudinal axis of the cavity is therefore smaller than the angle to a radial line orthogonal to the longitudinal axis and passing through the intersection. Substantially axial is understood in this context in particular to be when the connecting line of the centers of gravity of the two areas through which the fluid flows, which are spanned by the edges of the fluid inlet, deviates from the direction of the longitudinal axis of the cavity by less than 15 degrees.

The invention thus provides in particular a miniaturizable hollow cone nozzle (HKD) which can be manufactured in one piece from one piece of glass material.

By using glass material, preferably quartz glass or sapphire glass, a higher manufacturing quality can be achieved compared to the prior art. In particular, the surface quality inside the outlet channels and at the edges of their openings can be improved, which means that in mass production the achievable values for the atomization performance (dependent on the pressure loss) and the throughput of spray material are less scattered, defined spray properties can thus be guaranteed with smaller deviations.

Manufacture in suitable glass, in particular quartz glass or sapphire glass, described in more detail below, ensures exact adjustability of the (smallest) diameter of the fluid inlet and fluid outlet and an overall high production precision possible. Especially, a particularly good surface quality, i.e. low surface roughness, can be achieved using this material. Glass exposed to laser etching can be removed by using hydrofluoric acid (HF) or preferably caustic potash solution (KOH). The glass may also be suitably doped to improve laser processability. Such a manufacturing process is known per se under the name SLE (Selective, Laser-Induced Etching) and published in Hermans, M et al.: Selective, Laser-Induced Etching of Fused Silica at High Scan-Speeds Using KOH, JLMN-Journal of Laser Micro/Nanoengineering Vol. 9, No. 2, 2014. Production machines that can be used to manufacture using the SLE process are commercially available under the name LightFab Microscanner.

It is advantageous for the nozzle body to have one or more further fluid inlets which are predominantly tangential to the cavity jacket and which are arranged offset in the circumferential and/or axial direction.

According to one embodiment preferred in particular for achieving low flow rates, the smallest diameter of the fluid inlet is 50 micrometers or smaller, preferably 20 micrometers or smaller.

According to one embodiment preferred in particular for generating fine droplets, the smallest diameter of the fluid outlet is 50 micrometers or smaller, preferably 20 micrometers or smaller.

According to an advantageous enhancement, the nozzle body has a prechamber, wherein the fluid inlet can be fluidically flowed through from the prechamber to the cavity.

According to another advantageous enhancement, the nozzle body comprises a sieve body, which is formed in one piece with the rest of the nozzle body without a joint connecting the screen body and the rest of the nozzle body and preferably arranged on the inlet side outside the cavity.

The sieve body advantageously helps to avoid blockage of the fluid inlet or fluid outlet. Furthermore, a suitable choice of geometry, number and arrangement of the sieve openings of the sieve body provides additional degrees of freedom in the design of the nozzle body, for example to influence the pressure loss or the flow rate in a desired way.

Preferably, the sieve openings thereof are also manufactured by means of local laser exposure, in particular by subsequent etching away of material exposed to local laser exposure.

According to a particularly preferred embodiment, the fluid inlet can be fluidically flowed through from an antechamber to the cavity, and the sieve body forms a fluid inlet to the antechamber.

Preferably, the sieve body has only screen openings the respective smallest diameter of which (according to the above definition of a smallest diameter) is not larger than half the smallest diameter of the fluid inlet, particularly preferably not larger than one third of the smallest diameter of the fluid inlet.

Preferably, the sieve body is not thicker in the main flow-through direction of the sieve openings than five times the smallest diameter (according to the definition of a smallest diameter above) of the sieve openings (in case of screen openings of different sizes, of the smallest screen opening).

Also preferably, the total area of the sieve openings that can be flowed through is at least one hundred times the area of the fluid inlet that can be flowed through. The area openings that can be flowed through is an area orthogonal to the connecting line of the centres of gravity of the two flowed-through areas, which are spanned by the edges of the respective opening (sieve opening or fluid inlet), and is surrounded by the inner surface of the respective opening, in each case at the location of the respective smallest diameter.

According to another aspect, a spraying device is provided, which comprises a nozzle body according to one of the preceding claims. Furthermore, the spraying device has a spraying material supply device which supplies spraying material to the nozzle body, for example via a pipe or a pipe system or directly from a spraying material container, so that the latter combines the functions of storing and supplying spraying material. Valves, slides and other fluid retention and/or fluid flow regulating elements can be provided to regulate the pressure of the spray material the nozzle body is subjected to or the fluid flow output through the nozzle body.

In particular, as a spraying device a spray can is provided which has a spray head comprising a nozzle body of the type described above according to the invention and a container for the sprayed material. Such a spray can may advantageously be designed as a pump spray, so that a user, as known per se from the prior art, can exert the required fluid pressure in the sprayed material by means of a pumping movement, or else the sprayed material container contains a pressurized propellant, either in a common chamber with the sprayed material or separated therefrom by a piston, a membrane or the like. Other mechanical pressure build-up, in particular by means of spring systems, is also advantageously possible.

Advantageously, the spray head may have a spray head connecting member made of a plastic material, for example polyethylene, into which the nozzle body is inserted, which helps to keep production costs low. However, other materials, such as metal, can also be used for a spray head connecting member. The spray head connecting member can be designed as an injection moulded part at a particularly low cost.

According to a particularly advantageous embodiment, the spray head has adapter connection means for a patient airway adapter, wherein the spray head can be connected to the patient airway adapter by means of the adapter connection means without tools, for example by means of a plug or screw connection, in such a way that spray material escaping from the outlet channel can enter the patient airway adapter. Such a patient airway adapter may be designed as a nose adapter that can be inserted into a patient's nose, as a mouthpiece, or as a mask adapter covering a patient's mouth and/or a patient's nose.

The spray head may also advantageously be integrally formed or connected to a patient airway adapter, which in turn is configured as a nose adapter that can be inserted into a patient's nose, as a mask adapter covering a patient's mouth and/or a patient's nose, or as an adapter that can be enclosed by a patient's mouth.

Advantageously, the patient airway adapter may be made of a suitable plastic such as PE or PP, for example as an injection-molded part.

Preferably, the pressure in the spray container of the spray can does not exceed 30 bar, preferably it does not exceed 15 bar, in particular does preferably not exceed 10 bar. Even in the range of container pressures from 3 to 10 bar, the spray can according to the invention still achieves a much finer droplet size than conventional spray cans with a propellant.

For pressures of 6 bar and lower, especially 5 bar and lower, it has been found that, surprisingly for the skilled person, it is possible to achieve a further reduction of the droplet sizes achievable by spraying in this pressure range, if the smallest diameter of the fluid inlet and the smallest diameter of the fluid outlet is slightly larger than the above aspect of the invention, i.e. between 100 micrometers and 300 micrometers, preferably between 100 micrometers and 250 micrometers. While with reduced pressure the droplet diameters achievable by atomization become larger and in rages of higher pressure a reduction of the smallest outlet diameter usually results in a reduction of droplet size, in the pressure range below 6 bar, in particular below 5 bar, the increase of the possible droplet diameters in comparison with a higher pressure can be counteracted by somewhat increasing the smallest diameter (in particular the smallest diameter of the outlet), namely in the range from 100 micrometers to 300 micrometers, preferably from 100 micrometers to 250 micrometers.

Such a nozzle body made of glass material, which still belongs to the ultra-fine range, where conventional production technology reaches its limits, can be advantageously manufactured as described above and, apart from the enlarged fluid inlet and outlet diameter, be designed as described above. Again, using glass material, the present invention can achieve a higher production quality and a suitability for mass production that otherwise cannot be achieved economically in this scale.

Accordingly, in a further aspect, the present invention provides a spray device which comprises a nozzle body including a conical or cylindrical-conical cavity through which fluid can flow, which has a fluid inlet predominantly tangential relative to the cavity jacket and a fluid outlet substantially axial relative to the cavity jacket, the nozzle body being formed in one piece from a glass material, and the smallest diameter of the fluid inlet and the smallest diameter of the fluid outlet being between 100 micrometers and 300 micrometers, preferably between 100 micrometers and 250 micrometers, and which further comprises a spray material supply device for supplying spray material to the fluid inlet with a spray material pressure of 6 bar or less, preferably 5 bar or less.

The reduction of the achievable droplet sizes, which is surprising for the skilled person, by enlarging the smallest diameter of the fluid inlet and the fluid outlet to between 100 and 300 micrometers when the pressure of the spray material at the fluid inlet is below 6 bar, preferably below 5 bar, could be attributed to the fact that with finer inlet and outlet diameters, tulip formation of the spray material can take place inside the cavity when the smallest diameter of the fluid inlet and the fluid outlet is below 100 micrometers.

For the particularly preferred field of application of the spraying devices according to the present invention, these are charged or filled with at least one medicinal agent, preferably brine, and possibly additionally filled with a propellant. Even if the nozzle body according to the invention is mainly described for spray cans, especially for medical applications, the invention is not limited thereto. Rather, the nozzle body according to the present invention may be used in a variety of technical applications, in particular applications in which fine atomization at low volume flows and limited available pressure differences is desired, for example for the disinfection of air conditioning systems or compressed air-operated nebulizers for humidifying breathing air or skin surfaces in hospitals. A particularly fine atomization is also often desirable in cosmetic applications. Here, sprays with nozzle bodies according to the present invention can also be used to increase the product value.

According to another aspect, a medical inhaler is provided which has a nozzle body according to one of the preceding claims.

Herein-below, the invention is explained in more detail in an exemplary manner in connection with the accompanying schematic drawings. The drawings are not true to scale; in particular, for reasons of illustration, the ratios of the individual dimensions to one another do not always correspond to the ratios in actual technical implementations. Several preferred embodiments are described, but the invention is not limited thereto. In principle, any variant of the invention described or implied in the context of the present application may be particularly advantageous, depending on the economic, technical and, if applicable, medical conditions in an individual case. Unless otherwise stated, or if technically feasible, respectively, individual features of the described embodiments are interchangeable or can be combined with each other and with features known per se from the prior art.

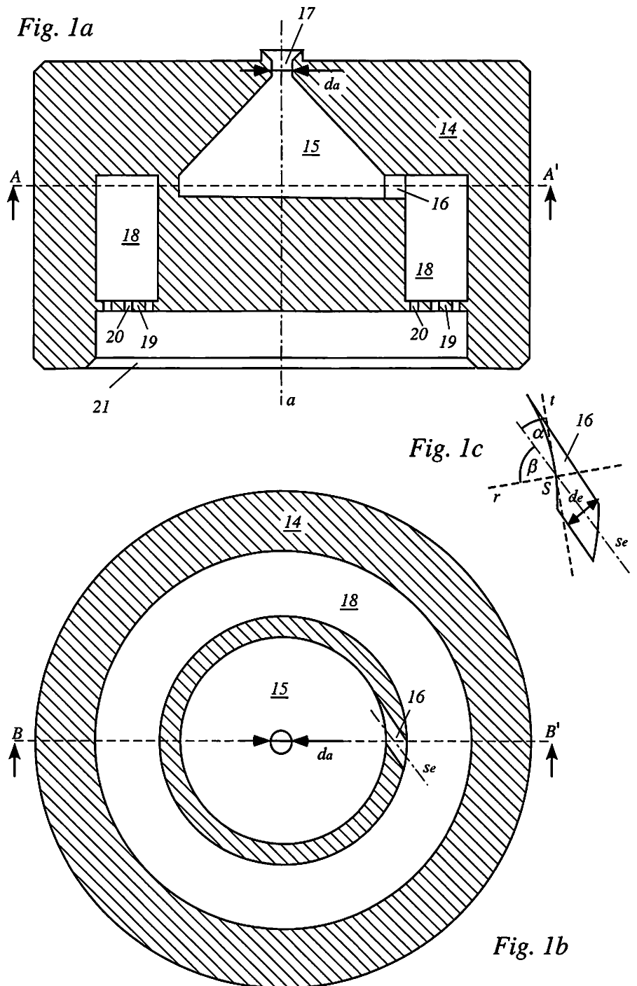

FIG. 1a shows a cross-sectional view of a nozzle body according to the invention, the sectional plane of FIG. 1b being indicated as a broken line A-A′,

FIG. 1b shows another cross-sectional view of the nozzle body of FIG. 1a, orthogonal to FIG. 1a, wherein the sectional plane of FIG. 1a is indicated as a broken line B-B′,

FIG. 1c shows the fluid inlet from FIG. 1b shows the fluid inlet from FIG. 1b as a detail to illustrate the geometric conditions,

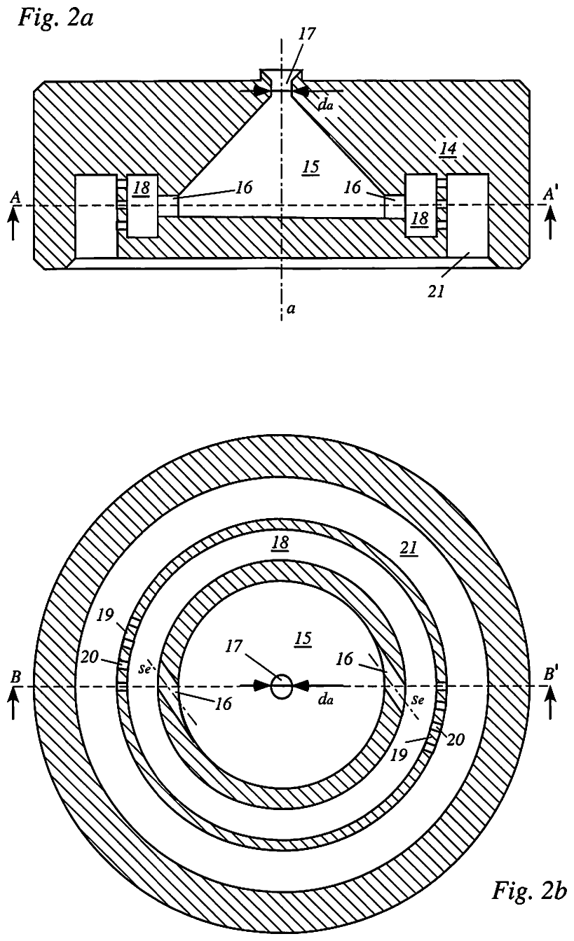

FIG. 2a shows a cross-sectional view of another nozzle body according to the invention, the sectional plane of FIG. 2b being indicated as a broken line A-A′,

FIG. 2b shows another cross-sectional view of the nozzle body of FIG. 2a, orthogonal to FIG. 2a, the sectional plane of FIG. 2a being indicated as a broken line B-B′, and

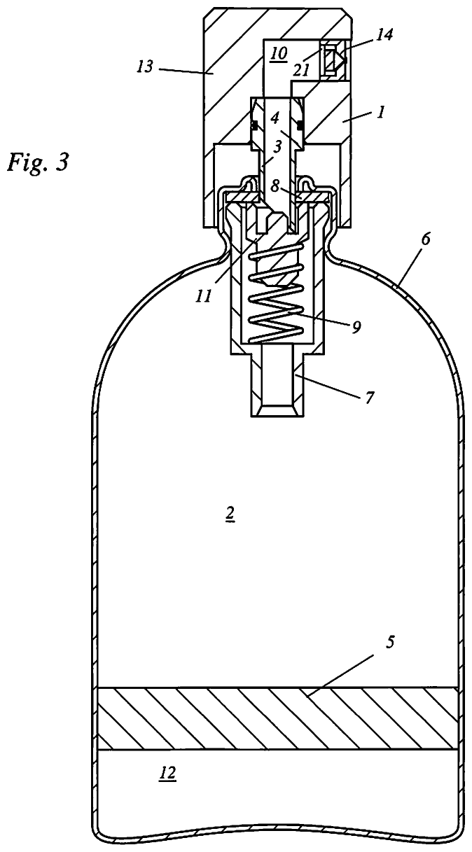

FIG. 3 shows a cross-section of a spray can according to the invention with a laterally discharging spray head.

Corresponding elements are marked with the same reference numerals in the figures of the drawings.

FIG. 1a shows a cross-sectional view of a nozzle body 14 according to the invention, wherein the sectional plane of FIG. 1b is indicated as a broken line A-N. The sectional plane of FIG. 1a is indicated in FIG. 1a as the broken line B-B′. The sectional planes of FIGS. 1a and 1b are therefore perpendicular to each other.

By means of selective laser exposure and subsequent etching of the exposed areas (selective laser-induced etching), a cylindrical-conical cavity 15 with a largely tangential fluid inlet 16 and an axial fluid outlet 17 is manufactured in the quartz glass nozzle body 14 with a cylindrical basic shape.

The fluid inlet 16 is shown enlarged in FIG. 1c. The axis of symmetry sₑ of the fluid inlet 16 connects the centers of gravity of the flowed through areas spanned by the edges of the fluid inlet 16. The angle α formed by the axis of symmetry sₑ and the line t tangent to the lateral surface of the cavity 15 is smaller than the angle β formed by the axis of symmetry sₑ and the radial straight line r orthogonal to the jacket surface at the intersection S with the tangent line t.

The smallest diameter of the fluid inlet 16 dₑ and fluid outlet 17 dₐ, respectively, is less than 100 μm. With the above-mentioned manufacturing method of selective laser-induced etching, even diameters of only a few micrometers can be achieved.

The axis of symmetry of the conical section of the cavity 15 is also the axis of symmetry of the fluid outlet 17. In the example shown, the fluid outlet 17 ends with a conical extension and a sharp tear-off edge to promote fluid atomization.

The liquid to be atomized is fed to the fluid inlet 16 via the inlet 21 and the annular antechamber 18. Between the inlet 21 and the antechamber 18, the liquid to be atomized flows through the (not necessarily provided) sieve body 19. The openings 20 thereof each have a minimum diameter of one third of the minimum diameter of the fluid inlet 16. The sieve body 19 prevents the fluid inlet 16 and the fluid outlet 17 from clogging.

There may also be several fluid inlets 16 distributed around the circumference, as shown in FIG. 2a. In this embodiment the sieve body 19 is arranged radially to the prechamber 18 and not axially, like the one shown in FIG. 1a. In contrast to FIG. 2b, instead of the circumferential prechamber 18, a separate prechamber may be provided for each fluid inlet 16.

The spray can in FIG. 2 comprises a spray head 1 in the spray head connecting part 13 of which the nozzle body 14 is inserted in such a way that the spray material is discharged to the side. Such an arrangement is advantageous for many technical (e.g. for disinfection) or cosmetic (e.g. for skin moistening or for the application of fragrances) applications.

The reusable spray head 1 is placed on the head 4 of the outlet pipe 3 of the spray container 2 and can be removed from it without tools in order to enable the replacement of the spray container 2. Alternatively, a configuration as a disposable product is also possible.

The spray tank 2 is designed as a two-chamber tank in which a movable piston 5 separates the compressed propellant, e.g. nitrogen, present in the lower area 12 of the spray tank 2 from the spray material, e.g. brine. Alternatively, the invention can also be implemented with a single-chamber container as spray container 2, in which the propellant and the sprayed material are mixed. The propellants known from conventional sprays are suitable as propellants. The container wall 6 of the spray tank 2 is made of conventional container material such as aluminum or tinplate.

The valve of the spray container 2 is similar to the valves of conventional spray cans and comprises a valve housing 7 which is sealed by the sealing ring 8 which consists of an elastic material such as rubber or silicone rubber. The spring 9 inserted in the valve housing presses the sealing cap 11 against the sealing ring 8. By pressing the spray head 1 and the spray container 2 together relative to each other, the outlet pipe 3, which is bevelled at the bottom, pushes the sealing cap downwards so that spray material can enter the feed channel 10 of the spray head 1 through the valve housing 7 and the outlet pipe 3.

The spray head connecting member 13 is made of a thermoplastic or thermosetting resin material, e.g. polyethylene, polypropylene or polycarbonate. The nozzle body 14, which is shown enlarged in FIGS. 1a and 1b, is inserted into the spray head connecting member 13.

1. A nozzle body comprising
a conical or cylindrical-conical cavity through which fluid can flow and which comprises a fluid inlet predominantly tangential relative to the cavity jacket and a fluid outlet substantially axial relative to the cavity jacket,
the nozzle body being formed in one piece from a glass material, and
the smallest diameter of the fluid inlet and the smallest diameter of the fluid outlet each being 100 micrometers or less.

2. The nozzle body according to claim 1, comprising another fluid inlet predominantly tangential relative to the cavity jacket.

3. The nozzle body according to claim 1, wherein the smallest diameter of the fluid inlet is 50 micrometers or less.

4. The nozzle body according to claim 1, wherein the smallest diameter of the fluid inlet is 20 micrometers or less.

5. The nozzle body according to claim 1, wherein the minimum diameter of the fluid outlet is 50 micrometers or less.

6. The nozzle body according to claim 1, wherein the minimum diameter of the fluid outlet is 20 micrometers or less.

7. The nozzle body according to claim 1, wherein the fluid inlet and fluid outlet are formed by means of local laser exposure.

8. The nozzle body according to claim 7, wherein the fluid inlet and the fluid outlet are formed by local laser exposure and subsequent etching away of material exposed to the local laser exposure.

9. The nozzle body according to claim 1, further comprising an antechamber, wherein the fluid inlet can be fluidically flowed through from the antechamber to the cavity.

10. The nozzle body according to claim 1, further comprising a sieve body formed together with the remaining nozzle body in one piece without a joint between the screen body and the remaining nozzle body.

11. The nozzle body according to claim 10, wherein the sieve body is arranged outside the cavity at the inlet side.

12. The nozzle body according to claim 10, wherein sieve openings in the sieve body are formed by means of local laser exposure.

13. The nozzle body according to claim 12, wherein the sieve openings in the screen body are formed by means of local laser exposure and subsequent etching away of material exposed to the local laser exposure.

14. The nozzle body according to claim 10, further comprising an antechamber, wherein the fluid inlet the fluid inlet can be fluidically flowed through from the antechamber to the cavity, and the sieve body forms a fluid inlet to the antechamber.

15. The nozzle body according to claim 10, wherein the sieve body has only sieve openings the respective smallest diameter of which is not greater than half the smallest diameter of the fluid inlet.

16. The nozzle body according to claim 15, wherein the sieve body has only sieve openings the respective smallest diameter of which is not greater than one-third of the smallest diameter of the fluid inlet.

17. The nozzle body according to claim 10, wherein the sieve body is not thicker than five times the smallest diameter of the sieve openings in the main flow direction through the sieve openings.

18. The nozzle body according to claim 10, wherein the total flowable area of the sieve openings is at least one hundred times the flowable area of the fluid inlet.

19. The nozzle body according to claim 1, wherein the glass material is quartz glass.

20. A spraying device comprising a nozzle body according to claim 1.

21. A spraying device comprising
a nozzle body which comprises a conical or cylindrical-conical cavity through which fluid can flow and which comprises a fluid inlet which is predominantly tangential relative to the cavity jacket and a fluid outlet which is substantially axial relative to the cavity jacket, wherein the nozzle body is formed in one piece from a glass material, and the smallest diameter of the fluid inlet and the smallest diameter of the fluid outlet is between 100 micrometers and 300 micrometers, preferably between 100 micrometers and 250 micrometers,
a spray material supply device for supplying spray material to the fluid inlet with a spray material pressure of 6 bar or less, preferably 5 bar or less.

22. The spraying device according to claim 20, said spraying device being configured as a spray can, comprising a spray material container and a spray head comprising the nozzle body.

23. The spraying device according to claim 22, said spraying device being configured as a pump spray.

24. The spraying device according to claim 22, wherein the spray material container contains a pressurized propellant.

25. The spraying device according to claim 20, said spraying device being configured as a medical inhaler.

26. A method for manufacturing, from a glass material, a nozzle body in one piece, which has a conical or cylindrical-conical cavity through which fluid can flow, wherein a, relative to the cavity jacket, predominantly tangential fluid inlet into the cavity and a, relative to the cavity jacket, substantially axial fluid outlet from the cavity are produced by local laser exposure and subsequent etching away of material exposed to the local laser exposure, such that the smallest diameter of the fluid inlet and the smallest diameter of the fluid outlet each are 100 micrometers or less.
